(19) United States
(12) Patent Application Publication
(10) Pub. No.: US 2024/0412862 A1
(43) Pub. Date: Dec. 12, 2024

(54) INFORMATION PROCESSING DEVICE, INFORMATION PROCESSING METHOD, AND STORAGE MEDIUM

(71) Applicant: CANON MEDICAL SYSTEMS CORPORATION, Otawara-shi (JP)

(72) Inventor: Akihiro TAGUCHI, Kawasaki (JP)

(73) Assignee: CANON MEDICAL SYSTEMS CORPORATION, Otawara-shi (JP)

(21) Appl. No.: 18/731,462

(22) Filed: Jun. 3, 2024

(30) Foreign Application Priority Data
Jun. 6, 2023 (JP) 2023-093357

(51) Int. Cl. G16H 40/67; G06F 3/01; G06F 3/16; G06T 19/00; G16H 10/60

(52) U.S. Cl. CPC G16H 40/67; G06F 3/011; G06F 3/167; G06T 19/00; G16H 10/60; G06T 2200/24; G06T 2219/024

(57) ABSTRACT

An information processing device includes processing circuitry. The processing circuitry acquires a motion history of a patient who represents themselves as an avatar and gets a check-up at a clinic in a metaverse. The processing circuitry determines a specific motion for estimating a state of the patient by analyzing the motion history. The processing circuitry outputs information based on the specific motion via an output interface.

### CROSS-REFERENCE TO RELATED APPLICATION

Priority is claimed on Japanese Patent Application No. 2023-093357, filed Jun. 6, 2023, the content of which is incorporated herein by reference.

### FIELD

Embodiments described in this specification and illustrated in the drawings relate to an information processing device, an information processing method, and a storage medium.

### BACKGROUND

A service called a metaverse clinic is provided. In this service, a user participates as an anonymous avatar in a metaverse and receives medical consultations, counselling, or the like with a qualified medical professional such as a doctor.

By using the metaverse, an effect such as a sense of staying together or a decrease in psychological barriers is obtained in addition to anonymity, and an effect of facilitated self-disclosure is obtained. Accordingly, a metaverse clinic is mainly utilized for patients with mental health problems.

As one final purpose of a patient with a metal health problem, it is conceivable that the patient gets a check-up at a clinic in the real world and comes into contact with a person in the real world. However, in a metaverse clinic provided to a patient with a metal health problem (that is, a metaverse mental clinic), since conversations are made between avatars, it may not be possible to reduce resistance to a clinic or a person in the real world.

### BRIEF DESCRIPTION OF THE DRAWINGS

FIG. 1 is a diagram illustrating an example of a configuration of an information processing system according to a first embodiment.

FIG. 2 is a diagram illustrating an example of a situation of an experience in a metaverse according to the first embodiment.

FIG. 3 is a diagram illustrating an example of a configuration of an information processing device according to the first embodiment.

FIG. 4 is a flowchart illustrating the flow of a series of processes that are performed by processing circuitry according to the first embodiment.

FIG. 5 is a diagram illustrating a method of determining a degree of return.

FIG. 6 is a diagram illustrating a method of determining a degree of return.

FIG. 7 is a diagram illustrating an example in which a degree of abstraction of a patient avatar is changed according to a degree of return.

FIG. 8 is a diagram illustrating an example in which a degree of abstraction of a patient avatar is changed according to a degree of return.

FIG. 9 is a diagram illustrating an example in which a patient with a low degree of return to the real world gets a check-up at a medical clinic.

FIG. 10 is a diagram illustrating an example in which a patient with a low degree of return to the real world gets a check-up at a medical clinic.

FIG. 11 is a flowchart illustrating a flow of a series of processes that are performed by processing circuitry according to a second embodiment.

FIG. 12 is a diagram illustrating an example in which a degree of abstraction of an external environment in the metaverse is changed according to a degree of return.

### DETAILED DESCRIPTION

Hereinafter, an information processing device, an information processing method, and a storage medium according to embodiments will be described with reference to the accompanying drawings.

An information processing device according to an embodiment includes processing circuitry. The processing circuitry acquires a motion history of a patient who represents themself as an avatar and gets a check-up at a clinic in a metaverse. The processing circuitry determines a specific motion for estimating a state of the patient by analyzing the motion history. The processing circuitry outputs information based on the specific motion via an output interface.

### First Embodiment

### [Configuration of Information Processing System]

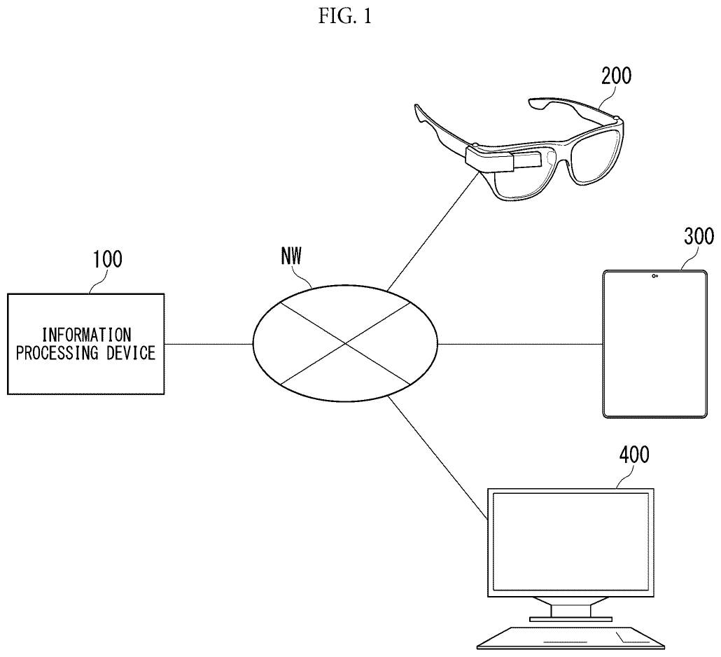

FIG. 1 is a diagram illustrating an example of a configuration of an information processing system 1 according to a first embodiment. The information processing system 1 according to the first embodiment includes, for example, an information processing device 100, a wearable device 200, a tablet terminal 300, and a laptop terminal 400. These devices are communicatively connected to each other via a communication network NW. The wearable device 200 is an example of an “output interface,” and the tablet terminal 300 and the laptop terminal 400 are other examples of the “output interface.”

“Communication network NW” refers to all information communication networks using electrical communication technology. The communication network NW may include a telephone communication network, an optical fiber communication network, a cable communication network, and a satellite communication network in addition to a local area network (LAN), a wide area network (WAN), and the Internet.

The information processing device 100 communicates with some or all of the wearable device 200, the tablet terminal 300, and the laptop terminal 400 via the communication network NW and collects a motion history of a patient P recorded by the device or terminal.

A patient P may be a patient who has represented themself as an avatar and gotten a check-up in a medical clinic (particularly, a psychosomatic clinic or a psychiatric clinic) in a metaverse (also referred to as a virtual space) in the past, a patient who represents themself as an avatar and is getting a check-up at a medical clinic, and/or a patient who represents themself as an avatar and is scheduled to get a check-up at the medical clinic. That is, a patient P is a patient suffering from a mental disease.

When a motion history of a patient P is collected from various devices or terminals, the information processing device 100 determines a physical and mental state of the patient P (specifically, a degree of return indicating the degree to which the patient P can return to the real world) by analyzing the motion history of the patient P,

Then, the information processing device 100 changes an output mode of an environment in the metaverse according to the physical and mental state of the patient P (the degree of return of the patient P to the real world).

The environment in the metaverse includes, for example, an appearance or a vocal sound of an avatar of a patient P (hereinafter referred to as a patient avatar A_{P}) and an appearance or a vocal sound of an avatar of a doctor (hereinafter referred to as a doctor avatar A_{D}) who has a conversation with the patient P at the time of diagnosing the patient P at the medical clinic. The patient avatar A_{P} is an example of a “first avatar,” and the doctor avatar A_{D} is an example of a “second avatar.”

The environment in the metaverse may include a landscape or a structure in the metaverse viewed from the patient avatar A_{P}. More specifically, the environment in the metaverse may include texture of a wall of the medical clinic, a room of the medical clinic or a chair or a table provided therein, a distance between the patient avatar A_{P} and the doctor avatar A_{D}, a piece of music played in the medical clinic, or various other external environmental factors.

The wearable device 200 is a device for experiencing the metaverse, and examples thereof include goggles, a headset, a head-mounted display, or smart glasses which are worn on the head for use. The wearable device 200 is, for example, a device using virtual reality (VR) technology. The wearable device 200 is not limited to VR and may be a device using other technology such as augmented reality (AR), mixed reality (MR), or projection mapping.

A controller or a glove that can operate an avatar in the metaverse may be accessory to the wearable device 200. The controller or the glove may be provided with a biometric sensor (for example, a pulse rate sensor, a perspiration sensor, or a temperature sensor), a motion sensor, a camera (a so-called in-camera) that can image a face of a patient P, or a microphone that can collect a vocal sound of the patient P. In this case, a detected value from the biometric sensor (such as a pulse rate, an amount of perspiration, or a body temperature), a detected value from the motion sensor (such as an acceleration or an angular velocity), an image captured by the camera (such as a face image of the patient P), or sound acquired by the microphone (such as a vocal sound of the patient P) is recorded as a part of the motion history of the patient P.

For example, a patient P may wear the wearable device 200, represent themself as a patient avatar A_{P} similar in appearance to the patient P or a patient avatar A_{P} similar to an animal or a character, and get a check-up at a medical clinic in the metaverse.

The tablet terminal 300 and the laptop terminal 400 are terminal devices (computers) which are used by a patient P. These devices may be used instead of the wearable device 200 or the controller or the glove accessory to the wearable device 200.

For example, the patient P may represent themself as a patient avatar A_{P} and get a check-up at a medical clinic in the metaverse using one or both of the tablet terminal 300 and the laptop terminal 400 instead of or in addition to use of the wearable device 200.

### [Situation of Experience in Metaverse]

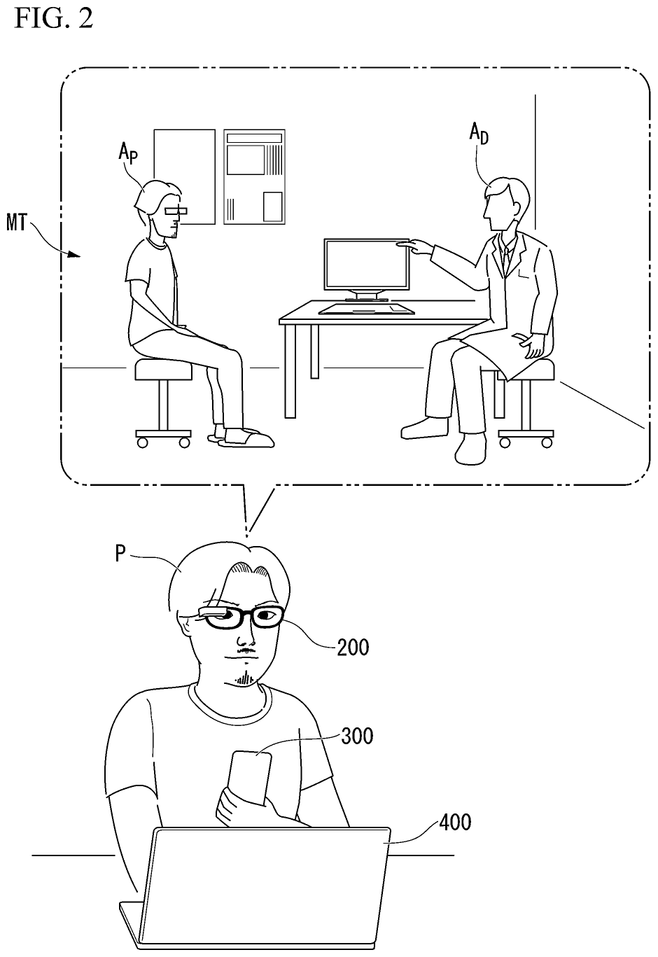

FIG. 2 is a diagram illustrating an example of a situation of an experience in the metaverse according to the first embodiment. MT in the drawing indicates a space in the metaverse, that is, a virtual space.

For example, a patient P wearing the wearable device 200 can represent themself as a patient avatar A_{P} and get a check-up at the medical clinic in the metaverse by seeing a video projected to the wearable device 200. At this time, the patient P can operate the patient avatar A_{P} in the metaverse by using the controller or the glove accessory to the wearable device 200 or using the tablet terminal 300 or the laptop terminal 400. For example, the patient P can operate the patient avatar A_{P} by operating a mouse or operating a touch panel. A mouse operation amount (such as an amount of movement or the number of clicks of a mouse pointer), a touch panel operation amount (for example, the number of taps or a scroll distance), or the like is recorded as a part of the motion history of the patient P.

### [Configuration of Information Processing Device]

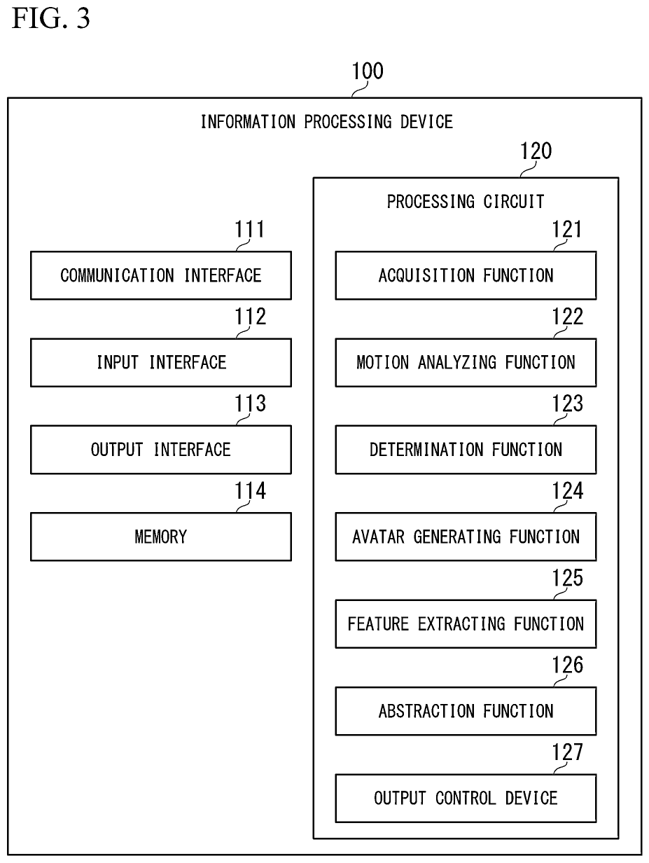

FIG. 3 is a diagram illustrating an example of a configuration of the information processing device 100 according to the first embodiment. The information processing device 100 includes, for example, a communication interface 111, an input interface 112, an output interface 113, a memory 114, and a processing circuit 120.

The communication interface 111 communicates with an external device via the communication network NW. Examples of the external device include the wearable device 200, the tablet terminal 300, and the laptop terminal 400. The communication interface 111 includes, for example, a network interface card (NIC) or a wireless communication antenna.

The input interface 112 receives various input operations from an operator, converts the received input operations to electrical signals, and outputs the electrical signals to the processing circuit 120.

For example, the input interface 112 includes a mouse, a keyboard, a trackball, a switch, a button, a joystick, or a touch panel. The input interface 112 may be, for example, a user interface for receiving a vocal input such as a microphone. When the input interface 112 is a touch panel, the input interface 112 may also have a display function of a display 113a included in the output interface 113 which will be described later.

The input interface 112 in this specification is not limited to physical operation components such as a mouse and a keyboard. For example, an electrical signal processing circuit that receives an electrical signal corresponding to an input operation from an external input device provided separately from the device and outputs the electrical signal to a control circuit is also included as an example of the input interface 112.

The output interface 113 includes, for example, a display 113a or a speaker 113b. The display 113a displays various types of information. For example, the display 113a displays an image generated by the processing circuit 120 or a graphical user interface (GUI) for receiving various input operations from an operator. For example, the display 113a is a liquid crystal display (LCD), a cathode ray tube (CRT) display, or an organic electroluminescence (EL) display. The speaker 113b outputs information input from the processing circuit 120 as a vocal sound.

The memory 114 is realized by, for example, a semiconductor memory element such as a random-access memory (RAM) or a flash memory, a hard disk, or an optical disc. These non-transitory storage media may be realized by different storage devices connected thereto via the communication network NW such as a network attached storage (NAS) or an external storage server.

The memory 114 may include a non-transitory storage medium such as a read only memory (ROM) or a register. A program which is executed by a hardware processor of the processing circuit 120, various calculation results from the processing circuit 120, the motion history of the patient P, and the like are stored in the memory 114.

The processing circuit 120 includes, for example, an acquisition function 121, a motion analyzing function 122, a determination function 123, an avatar generating function 124, a feature extracting function 125, an abstraction function 126, and an output control function 127. The acquisition function 121 is an example of an “acquisition unit,” the motion analyzing function 122 is an example of a “motion analyzing unit,” the determination function 123 is an example of a “return degree determining unit,” the abstraction function 126 is an example of an “abstraction unit,” and the output control function 127 is an example of an “output control unit.”

The processing circuit 120 realizes the functions, for example, by causing a hardware processor (a computer) to execute a program stored in the memory 114 (a storage circuit).

The hardware processor in the processing circuit 120 is, for example, circuitry such as a central processing unit (CPU), a graphics processing unit (GPU), an application-specific integrated circuit (ASIC), a programmable logic device (for example, a simple programmable logic device (SPLD) or a complex programmable logic device (CPLD)), or a field-programmable gate array (FPGA).

Instead of storing a program in the memory 114, the program may be directly input to a circuit of the hardware processor. In this case, the hardware processor realizes the functions by reading and executing the program input to the circuit. The program may be stored in the memory 114 in advance, or may be stored in a non-transitory storage medium such as a DVD or a CD-ROM and installed in the memory 114 from the non-transitory storage medium by setting the non-transitory storage medium into a drive device (not illustrated) of the information processing device 100. The hardware processor is not limited to a single circuit, but may be configured as one hardware processor into which a plurality of independent circuits are combined to realize the functions. A plurality of elements may be combined into one hardware processor to realize the functions.

### [Process Flow in Information Processing Device]

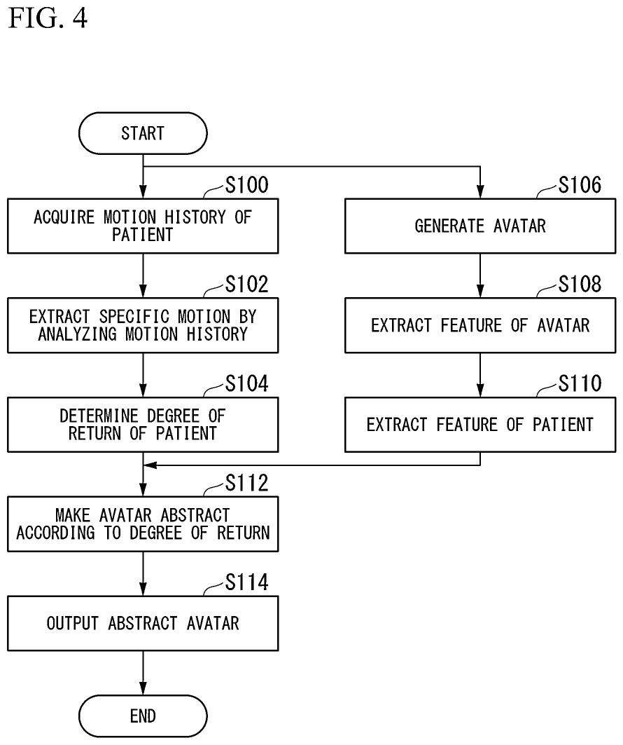

A series of processes that are performed by the processing circuit 120 of the information processing device 100 will be described below with reference to a flowchart. FIG. 4 is a flowchart illustrating a flow of a series of processes that are performed by the processing circuit 120 according to the first embodiment.

First, the acquisition function 121 acquires a motion history of a patient P (Step S100).

As described above, the patient P is a patient who gets (has gotten or will get) a check-up at a medical clinic at one or more time points in the past, the present, or the future. Accordingly, the motion history of the patient P includes a motion history at one or more time points in the past, the present, or the future.

More specifically, the motion history of the patient P includes at least one of a motion history before the patient P gets a check-up at the medical clinic, a motion history after the patient P has gotten a check-up at the medical clinic, and a motion history when the patient P is getting a check-up at the medical clinic.

These motion histories include motions which are performed by the patient P in the metaverse or motions which are performed by the patient P in the real world.

The motion history of the patient P includes, for example, a motion of the patient avatar A_{P} operated by the patient P, a motion of the patient P when the patient avatar A_{P} is operated, a speech of the patient P, a motion in the metaverse, and a motion in the world. Some thereof will be described below, but the motion history is not limited thereto.
(i) Motions of a patient avatar A_{P} (motions of a patient avatar A_{P} operated by a patient P)
   Positive expressions (such as liking, laughter, and bowing)
   Negative expressions (such as sadness, fear, and sorrow)
   Emotional expressions (such as curiosity, happiness, pleasance, or anger)
   Basic operations in the metaverse (such as standing, sitting, walking, and running)
(ii) Motions of a patient P detected by the motion sensor, the biometric sensor, the camera, or the microphone (motions of a patient P when a patient avatar A_{P} is operated)
   Number of performed motions, such as blinking, gazing, nodding, leg shaking, taking-off of the wearable device 200, standing, sitting, mouse motion, sound volume, expression, heartbeat, brain waves, and the like
(iii) Speech of patient P
   Speech details, words, sentences, context, or questions when a patient P has a conversation with a doctor or someone else
(iv) Other motions
   Number of uses of chatting, number or time of conversations, time in the metaverse (login time), number of Internet searches performed by a doctor who has provided a check-up, number of Internet searches performed at a clinic at which a check-up has taken place, and the like

For example, the acquisition function 121 may access the wearable device 200, the tablet terminal 300, or the laptop terminal 400 via the communication interface 111 and acquire a motion history of a patient P therefrom. When a motion history of a patient P is input to the input interface 112, the acquisition function 121 may acquire the input motion history of the patient P from the input interface 112. When a motion history of a patient P is stored in the memory 114, the acquisition function 121 may acquire the motion history of the patient P from the memory 114.

Then, the motion analyzing function 122 extracts or determines a specific motion for estimating a state of a patient P by analyzing the motion history of the patient P acquired by the acquisition function 121 (Step S102).

For example, the motion analyzing function 122 extracts a self-disclosure motion as the specific motion out of a series of motions of the patient P included in the motion history.

It has been recognized through many studies that there is a likelihood that self-disclosure will cause recovery of mental health. (i) A hobby or a preference, (ii) an awkward experience, (iii) a defect or weakness, and (iv) a negative in character or ability have been reported as types of the self-disclosure.

For example, the motion analyzing function 122 extracts an index (a self-disclosure motion) for quantitatively calculating each of the four types of self-disclosure out of a series of motions of the patient P included in the motion history. Some indices will be described below, and the present invention is not limited thereto.
(a) How much a patient can talk
   Number of times a word associated with self-disclosure has been spoken
   Time in which speech for self-disclosure has been made
   Type of self-disclosure, and the like
(b) Has a question been received from an opponent?
   Backchannel
   Number of questions
   Time in which speech has been heard
   Number of times a positive or negative expression (an avatar action) has been used
   Number of times a doctor has been searched for, and the like
(c) How much stress has been applied?
   Number or time of eye contact
   Number of times avatar actions have been performed (time in which the controller has been operated)
   Heartbeat
   Change in sound volume or tone
   Amount of perspiration, and the like

Then, the determination function 123 determines or calculates a degree of return of the patient P to the real world on the basis of the self-disclosure motion extracted as an index by the motion analyzing function 122 (Step S104).

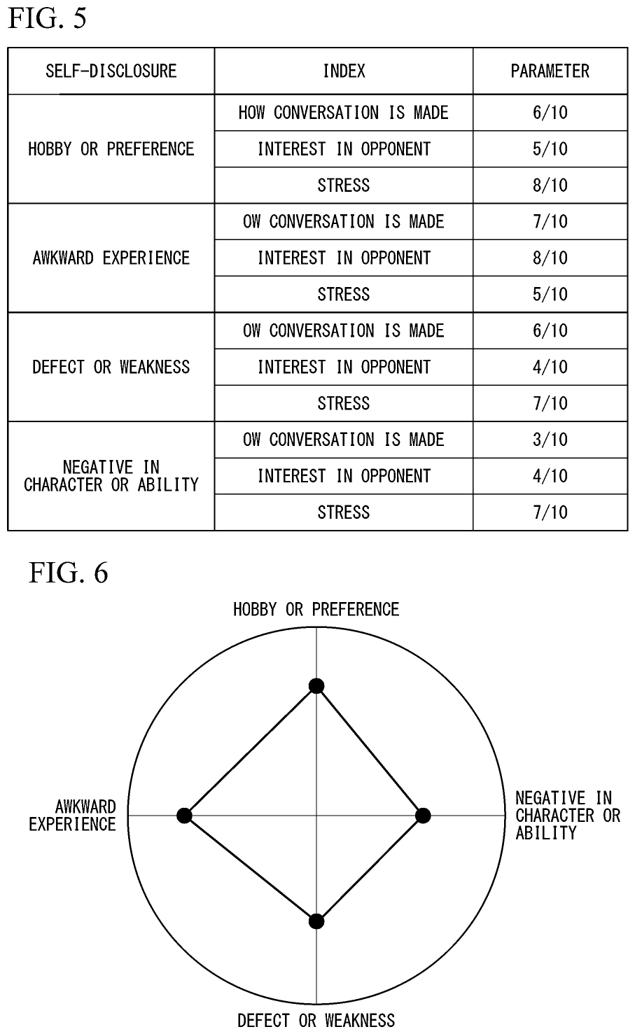

FIGS. 5 and 6 are diagrams illustrating a method of determining a degree of return. For example, the determination function 123 parameterizes (digitizes) the indices (motions) of (a) to (c) for each of four types of self-disclosure of (i) to (iv) as illustrated in FIG. 5. The upper limit of each parameter is determined, and the value thereof can be determined according to the number of motions or the time associated with each type of self-disclosure.

The determination function 123 performs statistical calculation (for example, averaging) of the parameters of all the indices of (a) to (c) on the same type of self-disclosure as (i) a hobby or a preference and calculates one representative parameter. Similarly, the determination function 123 performs statistical calculation of the parameters of all the indices of (a) to (c) on the same type of self-disclosure as (ii) an awkward experience and calculates one representative parameter. The determination function 123 performs statistical calculation of the parameters of all the indices of (a) to (c) on the same type of self-disclosure as (iii) a defect or a weakness and calculates one representative parameter. The determination function 123 performs statistical calculation of the parameters of all the indices of (a) to (c) on the same type of self-disclosure as (iv) a negative in character or ability and calculates one representative parameter. Accordingly, four-dimensional parameters (that is, four-dimensional vectors) are generated as illustrated in the radar chart of FIG. 6.

The determination function 123 calculates the sum of the four-dimensional parameters as the degree of return of the patient P to the real world. That is, the determination function 123 sums the representative parameter associated with the same type of self-disclosure as (i) the hobby or preference, the representative parameter associated with the same type of self-disclosure as (ii) the awkward experience, the representative parameter associated with the same type of self-disclosure as (iii) the defect or weakness, and the representative parameter associated with the same type of self-disclosure as (iv) the negative in character or ability and calculates the summed value as the degree of return of the patient P to the real world.

The determination function 123 may calculate the degree of return of the patient P to the real world, for example, by parameterizing the indices (motions) of (a) to (c) for each emotion (joy, anger, sorrow, and pleasure) in addition to or instead of the self-disclosure.

Description will be continued with reference back to the flowchart. The avatar generating function 124 generates an avatar that is operated by each user in the metaverse in parallel with the process of S100 (Step S106). This avatar includes the patient avatar A_{P} or the doctor avatar A_{D}.

The avatar may be typically a three-dimensional avatar. The three-dimensional avatar includes information on a surface shape of a three-dimensional structure (so-called “polygon”), information on an internal framework (so-called “bone”), information for defining a relationship therebetween (so-called “weight”), and information on texture or shape on the surface of the three-dimensional structure (so-called “texture”). The three-dimensional avatar in this embodiment may include all such information, may include only the polygon and the texture, or may include only the polygon. The avatar is not limited to a three-dimensional avatar, but may be a two-dimensional avatar.

For example, the avatar generating function 124 may generate an avatar similar to a person in the real world (that is, a patient P or a doctor), generate an avatar similar to an animal in the real world (for example, a dog or a cat), or generate an avatar imitating an imaginary or virtual character or life which is not present in the real world. For example, what avatar to generate may be arbitrarily determined by each user participating in the metaverse.

Then, the feature extracting function 125 extracts features of the avatar (for example, the patient avatar A_{P}) generated by the avatar generating function 124 (Step S108).

For example, when the avatar generated by the avatar generating function 124 is a humanoid avatar, the feature extracting function 125 extracts an eye, a nose, a mouth, a body type or physique, texture, and the like as features.

For example, when the avatar generated by the avatar generating function 124 is an avatar of an animal or a creature, the feature extracting function 125 extracts unique features (for example, ivory, a horn, and ears) of the avatar.

Then, the feature extracting function 125 extracts features of a user (for example, the patient P) serving as the origin of the avatar (Step S110).

For example, the feature extracting function 125 extracts eyes, a nose, a mouth, a body type or physique, a skin color, and the like of the user as features.

Then, the abstraction function 126 makes the avatar generated by the avatar generating function 124 abstract according to the degree of return of the patient P to the real world (Step S112).

Then, the output control function 127 transmits the avatar (which includes an abstract avatar) to at least one of the wearable device 200, the tablet terminal 300, and the laptop terminal 400 which are external devices via the communication interface 111 (Step S114). Accordingly, the wearable device 200, the tablet terminal 300, and/or the laptop terminal 400 displays the avatar in the metaverse.

Abstraction means that features of a user are reflected in an avatar while original features (default features) of the avatar is left according to the degree of return of the patient P to the real world. In other words, abstraction means that the original features (default features) of the avatar are made to approach the features of the user according to the degree of return of the patient P to the real world. The approached features may be partial features such as eyes, a nose, and a mouth or may be the whole face or body thereof.

For example, when the degree of return changes in a numerical value of 0 to 100, a degree of abstraction of the avatar may be determined by Expression (1).

{(100-degree of return)×(feature of avatar)+(degree of return)×(feature of user)}/100 (1)

For example, the abstraction function 126 changes the appearance of the patient avatar A_{P} such that it is less like the appearance of the patient P in the real world as the degree of return of the patient P to the real world becomes lower and changes the appearance of the patient avatar A_{P} such that it is more like the appearance of the patient P in the real world as the degree of return of the patient P to the real world becomes higher.

In other words, the abstraction function 126 increases the degree of abstraction of the appearance of the patient avatar A_{P} such that it is less like the appearance of the patient P in the real world as the degree of return of the patient P to the real world becomes lower and decreases the degree of abstraction of the appearance of the patient avatar A_{P} such that it is more like the appearance of the patient P in the real world as the degree of return of the patient P to the real world becomes higher.

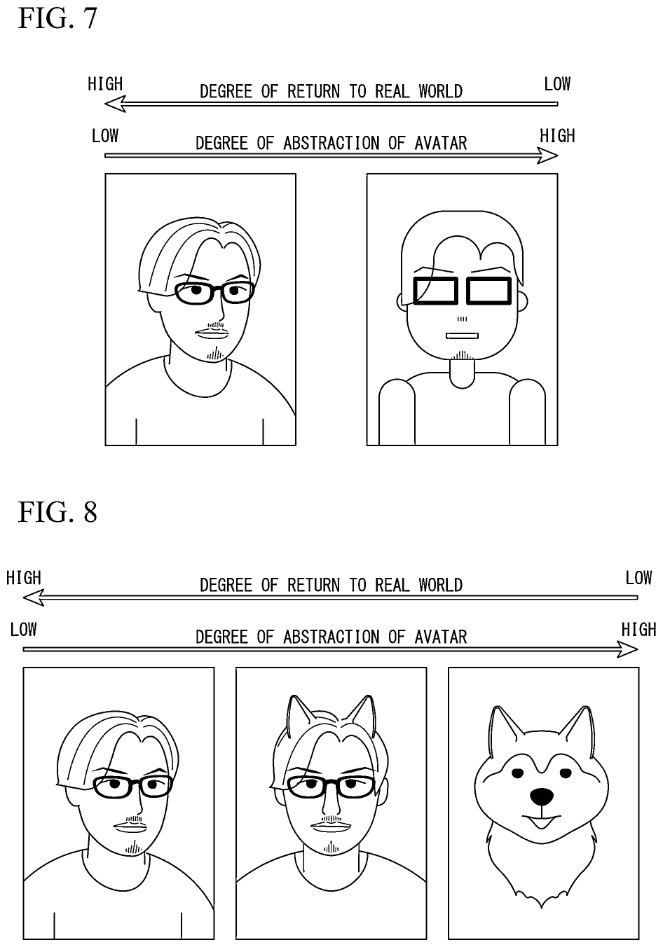

FIG. 7 is a diagram illustrating an example in which a degree of abstraction of a patient avatar A_{P} is changed according to a degree of return. In the illustrated example, it is assumed that the patient avatar A_{P} generated by the avatar generating function 124 is a humanoid avatar and basic features of the patient P such as a hairstyle, whether the avatar wears glasses, and whether the avatar has beard, or the like are reflected in the patient avatar A_{P} in advance.

As illustrated in the drawing, since the features of the patient P becomes more reflected in the patient avatar A_{P} as the degree of return of the patient P to the real world becomes higher, the appearance of the patient avatar A_{P} changes gradually from an animation tone to a real tone. In this way, when the degree of return of the patient P to the real world is low and the patient is in a psychological state in which the patient easily feels psychological resistance, the appearance of the patient avatar A_{P} can be made to be less like the appearance of the patient P in the real world, and thus it is possible to reduce psychological resistance of the patient P with respect to a medical clinic, a doctor, or the like when the patient gets a check-up in the medical clinic in the metaverse.

FIG. 8 is a diagram illustrating an example in which a degree of abstraction of a patient avatar A_{P} is changed according to a degree of return. In the illustrated example, it is assumed that the patient avatar A_{P} generated by the avatar generating function 124 is a dog-shaped avatar and none of basic features of the patient P such as a hairstyle, whether the avatar wears glasses, and whether the avatar has beard, or the like are reflected in the patient avatar A_{P} in advance.

As illustrated in the drawing, since the features of the patient P become more reflected in the patient avatar A_{P} as the degree of return of the patient P to the real world becomes higher, a full dog-shaped avatar changes gradually to a semi-humanoid avatar and changes finally to a humanoid avatar. In this way, when the degree of return of the patient P to the real world is low and the patient is in a psychological state in which the patient sensitive to psychological resistance, the appearance of the patient avatar A_{P} can be made to be a dog shape completely different from the appearance of the patient P in the real world. As a result, it is possible to reduce psychological resistance of the patient P with respect to a medical clinic, a doctor, or the like when the patient gets a check-up in the medical clinic in the metaverse.

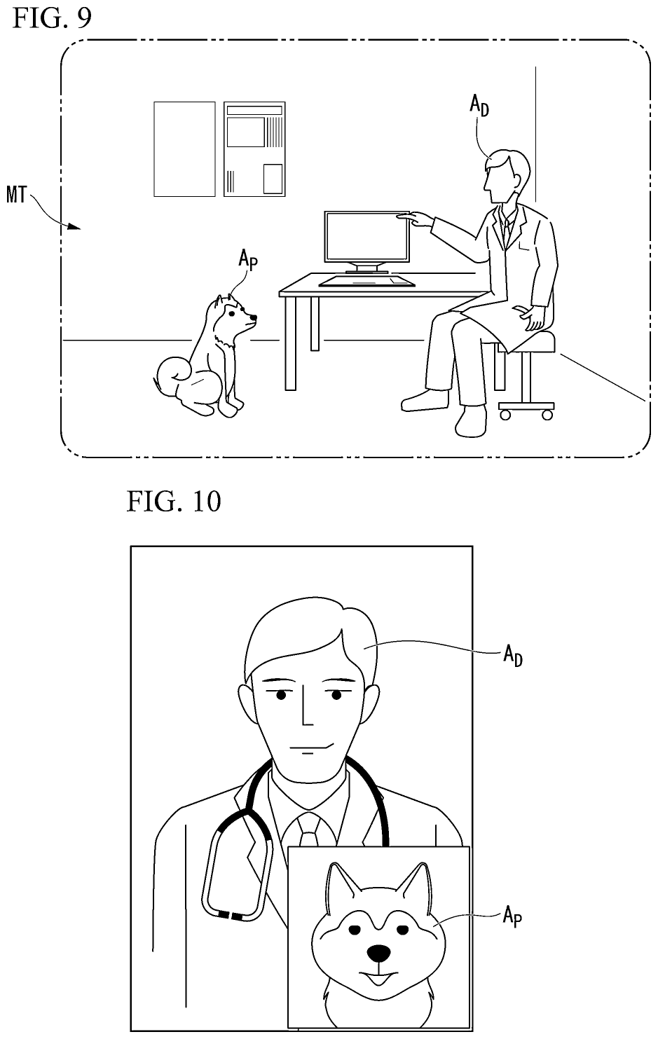

FIG. 9 is a diagram illustrating an example in which a patient P with a low degree of return to the real world gets a check-up at a medical clinic. As illustrated in the drawing, a patient P with a low degree of return to the real world can represent themselves as a patient avatar A_{P} of a complete dog shape (conceals the appearance thereof) and make a conversation with a doctor avatar A_{D}. As illustrated in the drawing, when a conversation with the doctor avatar A_{D} in the metaverse is made at the third-person viewpoint, the patient P can get a check-up while ascertaining the appearance of the patient avatar A_{P} at the third-person viewpoint.

FIG. 10 is a diagram illustrating an example in which a patient P with a low degree of return to the real world gets a check-up at a medical clinic. As illustrated in the drawing, when a conversation with a doctor avatar A_{D} in the metaverse is made at the first-person viewpoint, the patient avatar A_{P} may be superimposed and displayed on the doctor avatar A_{D} using a technique such as chroma-key synthesis. Accordingly, the patient P can get a check-up while ascertaining the appearance of the patient avatar A_{P} even at the first-person viewpoint.

According to the first embodiment described above, the processing circuit 120 of the medical information processing device 100 acquires a motion history of a patient P which represents themselves as a patient avatar A_{P} and gets a check-up at a medical clinic in the metaverse. The processing circuit 120 determines a specific motion for estimating the state of the patient P by analyzing a motion history of the patient P. The processing circuit 120 determines a degree of return of the patient P to the real world on the basis of the specific motion. The processing circuit 120 makes the patient avatar A_{P} abstract according to the degree of return of the patient P to the real world. Accordingly, when a patient P gets a check-up at a medical clinic in the metaverse, it is possible to reduce the psychological resistance of the patient P with respect to the medical clinic, a doctor, or the like.

### Modified Examples of First Embodiment

Modified examples of the first embodiment will be described below. For example, the abstraction function 126 may make a vocal sound of the patient avatar A_{P} abstract in addition to the figure (appearance) of the patient avatar A_{P} according to the degree of return of the patient P to the real world.

For example, the abstraction function 126 changes the vocal sound of the patient avatar A_{P} such that the vocal sound is less like a vocal sound of the patient P in the real world as the degree of return of the patient P to the real world becomes lower and changes the vocal sound of the patient avatar A_{P} such that the vocal sound is more like the vocal sound of the patient P in the real world as the degree of return of the patient P to the real world becomes higher.

That is, the abstraction function 126 increases a degree of abstraction of the vocal sound of the patient avatar A_{P} such that the vocal sound is less like the vocal sound of the patient P in the real world as the degree of return of the patient P to the real world becomes lower and decreases the degree of abstraction of the vocal sound of the patient avatar A_{P} such that the vocal sound is more like the vocal sound of the patient P in the real world as the degree of return of the patient P to the real world becomes higher.

The abstraction function 126 may make an appearance or a vocal sound of a doctor avatar A_{P} in addition to or instead of the figure (appearance) or the vocal sound of the patient avatar A_{P} according to the degree of return of the patient P to the real world.

For example, the abstraction function 126 changes the appearance or the vocal sound of the doctor avatar A_{D} such that the appearance or the vocal sound is less like an appearance or a vocal sound of a doctor in the real world as the degree of return of the patient P to the real world becomes lower and changes the appearance or the vocal sound of the doctor avatar A_{D} such that the appearance or the vocal sound is more like the appearance or the vocal sound of the doctor in the real world as the degree of return of the patient P to the real world becomes higher.

In other words, the abstraction function 126 increases a degree of abstraction of the appearance or the vocal sound of the doctor avatar A_{D} such that the appearance or the vocal sound is less like the appearance or the vocal sound of the doctor in the real world as the degree of return of the patient P to the real world becomes lower and decreases the degree of abstraction of the appearance or the vocal sound of the doctor avatar A_{D} such that the appearance or the vocal sound is more like the appearance or the vocal sound of the doctor in the real world as the degree of return of the patient P to the real world becomes higher.

The abstraction function 126 may change the appearance or the vocal sound of the patient avatar A_{P} or the appearance or the vocal sound of the doctor avatar A_{D} using the degree of return determined by the doctor in addition to or instead of the degree of return determined by the determination function 123. For example, it is assumed that the communication interface 111 receives the degree of return independently determined by the doctor or the degree of return independently determined by the doctor is input to the input interface 112. In this case, the abstraction function 126 may change the appearance or the vocal sound of the patient avatar A_{P} or the appearance or the vocal sound of the doctor avatar A_{D} using the degree of return received via the communication interface 111 or the degree of return input to the input interface 112.

The determination function 123 may decrease or increase the degree of return according to a fluctuation width in emotion of the patient P when calculating the degree of return of the patient P to the real world. For example, when an emotion of pleasance and an emotion of sadness appear alternately in the motion history of the patient P and the emotion of the patient P is not consistency, the determination function 123 may decrease the degree of return.

### Second Embodiment

A second embodiment will be described below. In the first embodiment, the appearance or the vocal sound of the patient avatar A_{P} or the appearance or the vocal sound of the doctor avatar A_{D} are made to be abstract according to the degree of return of the patient P to the real world. On the other hand, the second embodiment is different from the first embodiment, in that an external environment (such as a landscape) in the metaverse viewed from the patient avatar A_{P} is made to be abstract according to the degree of return of the patient P to the real world. In the following description, differences from the first embodiment will be mainly described, and the same description as in the first embodiment will be omitted. The same elements in the second embodiment as in the first embodiment will be referred to by the same reference signs, and description thereof will be omitted.

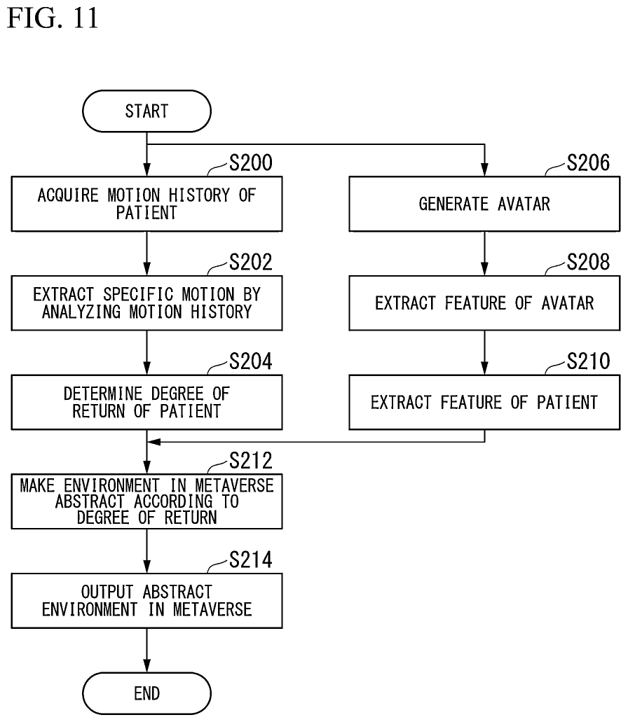

FIG. 11 is a flowchart illustrating a process flow that is performed by the processing circuit 120 according to the second embodiment.

First, the acquisition function 121 acquires a motion history of a patient P (Step S200).

Then, the motion analyzing function 122 extracts or determines a specific motion for estimating a state of the patient P, that is, a self-disclosure motion, by analyzing the motion history of the patient P acquired by the acquisition function 121 (Step S202).

Then, the determination function 123 determines or calculates a degree of return of the patient P to the real world on the basis of the self-disclosure motion extracted by the motion analyzing function 122 (Step S204).

Then, the avatar generating function 124 generates an avatar that is operated by a user in the metaverse or a virtual space (a virtual space in which the avatar is disposed) similar to a room of a medical clinic in the real world in parallel with the process of S200 (Step S206).

Then, the feature extracting function 125 extracts features of the avatar (for example, a patient avatar A_{P}) generated by the avatar generating function 124 or features of the virtual space similar to the room of the medical clinic in the real world (Step S208).

Then, the feature extracting function 125 extracts features of a user (for example, a patient P) serving as an origin of the avatar or features of the room of the medical clinic in the real world (Step S210).

Then, the abstraction function 126 makes an external environment in the metaverse viewed from the patient avatar A_{P} abstract according to the degree of return of the patient P to the real world (Step S212).

The external environment in the metaverse includes, for example, a color or texture of a wall of the room of the medical clinic, a chair or a table disposed in the room, and a piece of music played in the room in addition the doctor avatar A_{D}.

Then, the output control function 127 transmits the patient avatar A_{P} or the external environment to at least one of the wearable device 200, the tablet terminal 300, and the laptop terminal 400 which are external devices via the communication interface 111 (Step S114). Accordingly, the wearable device 200, the tablet terminal 300, and/or the laptop terminal 400 display the avatar or the medical clinic in the metaverse.

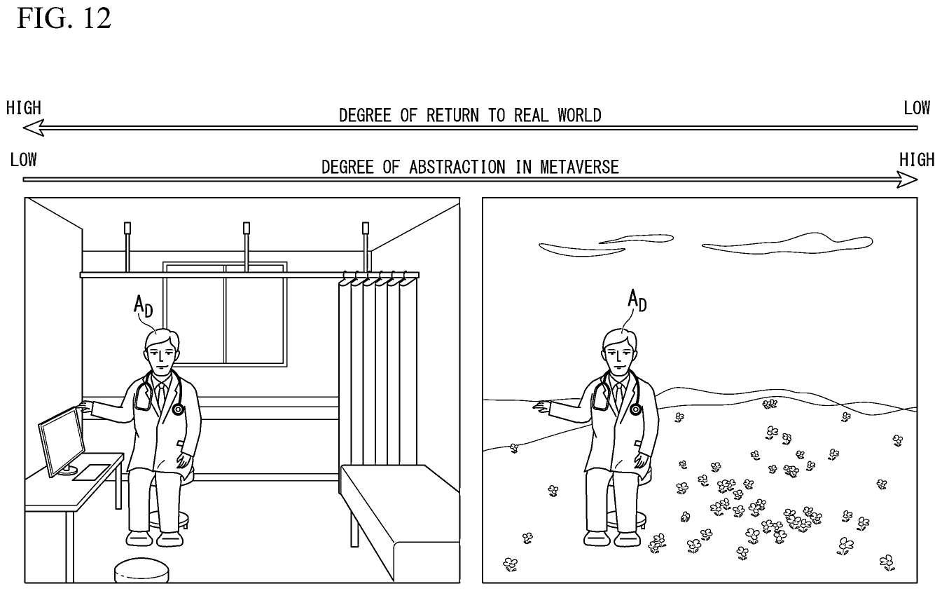

FIG. 12 is a diagram illustrating an example in which a degree of abstraction of the external environment in the metaverse is changed according to a degree of return. For example, the abstraction function 126 changes the external environment in the metaverse such that the external environment is less like the medical clinic in the real world as the degree of return of the patient P to the real world becomes lower and changes the external environment in the metaverse such that the external environment is more like the medical clinic in the real world as the degree of return of the patient P to the real world becomes higher.

In other words, the abstraction function 126 increases the degree of abstraction of the external environment in the metaverse such that the degree of abstraction is less like the medical clinic in the real world as the degree of return of the patient P to the real world becomes lower and changes the degree of abstraction of the external environment in the metaverse such that the degree of abstraction is more like the medical clinic in the real world as the degree of return of the patient P to the real world becomes higher.

Accordingly, it is possible to display a landscape which cannot be present in the room of the medical clinic in the real world as texture when the degree of return of the patient P to the real world is low as illustrated in the drawing, and it is possible to display a landscape more like the room of the medical clinic as texture as the degree of return of the patient P to the real world becomes higher. In this way, since the external environment in the metaverse is made to be less like the real world when the degree of return of the patient P to the real world is low and the patient is in a psychological state in which the patient is sensitive to psychological resistance, it is possible to reduce psychological resistance of the patient P with respect to a medical clinic, a doctor, or the like when the patient gest a check-up at the medical clinic in the metaverse.

According to the second embodiment described above, the processing circuit 120 of the medical information processing device 100 acquires a motion history of a patient P which represents themselves as a patient avatar A_{P} and gets a check-up at a medical clinic in the metaverse. The processing circuit 120 determines a specific motion for estimating the state of the patient P by analyzing the motion history of the patient P. The processing circuit 120 determines a degree of return of the patient P to the real world on the basis of the specific motion. The processing circuit 120 makes the external environment in the metaverse viewed from the patient avatar A_{P} abstract according to the degree of return of the patient P to the real world. Accordingly, when a patient P gets a check-up at a medical clinic in the metaverse, it is possible to reduce the psychological resistance of the patient P with respect to the medical clinic, a doctor, or the like.

### Modified Examples of Second Embodiment

Modified examples of the second embodiment will be described below. For example, the abstraction function 126 may change a piece of music played in a room of a medical clinic in addition to texture of the room according to the degree of return of the patient P to the real world.

The abstraction function 126 may change a distance between a patient avatar A_{P} and a doctor avatar A_{D} disposed in the room or an area of the room in addition to the texture of the room of the medical clinic or the piece of music played in the room according to the degree of return of the patient P to the real world.

For example, the abstraction function 126 may increase the distance between the patient avatar A_{P} and the doctor avatar A_{D} as the degree of return of the patient P to the real world becomes lower and decrease the distance between the patient avatar A_{P} and the doctor avatar A_{D} as the degree of return of the patient P to the real world becomes higher.

For example, the abstraction function 126 may increase the area of the room of the medical clinic formed in the metaverse as the degree of return of the patient P to the real world becomes lower and decrease the area of the room of the medical clinic formed in the metaverse as the degree of return of the patient P to the real world becomes higher.

While some embodiments have been described above, these embodiments are provided as examples and are not intended to limit the scope of the present invention. These embodiments can be realized in various other forms, and various omissions, substitutions, and modifications can be added thereto without departing from the gist of the present invention. These embodiments and modifications thereof are included in the scope or gist of the present invention and are also included in the inventions described in the appended claims and equivalent scopes thereof.

What is claimed is:

1. An information processing device comprising processing circuitry, the processing circuitry performing:
acquiring a motion history of a patient who represents themself as an avatar and gets a check-up at a clinic in a metaverse;
determining a specific motion for estimating a state of the patient by analyzing the motion history; and
outputting information based on the specific motion via an output interface.

2. The information processing device according to claim 1, wherein the motion history includes one or both of a motion history of the patient in the metaverse and a motion history of the patient in the real world.

3. The information processing device according to claim 1, wherein the motion history includes at least one of a motion history before the patient gets a check-up at the clinic, a motion history after the patient has gotten a check-up at the clinic, and a motion history when the patient is getting a check-up at the clinic.

4. The information processing device according to claim 1, wherein the processing circuitry performs:
determining a degree of return indicating a degree to which the patient is able to return to the real world on the basis of the specific motion; and
outputting information based on the degree of return via the output interface.

5. The information processing device according to claim 4, wherein the processing circuitry performs:
outputting a first avatar which is an avatar of the patient in the metaverse via the output interface; and
making an appearance of the first avatar in the metaverse abstract according to the degree of return.

6. The information processing device according to claim 5, wherein the processing circuitry changes the appearance of the first avatar such that the appearance is less like a real appearance of the patient as the degree of return becomes lower and changes the appearance of the first avatar such that the appearance is more like the real appearance of the patient as the degree of return becomes higher.

7. The information processing device according to claim 5, wherein the processing circuitry performs:
additionally outputting a vocal sound of the first avatar via the output interface; and
changing the vocal sound of the first avatar in the metaverse according to the degree of return.

8. The information processing device according to claim 7, wherein the processing circuitry changes the vocal sound of the first avatar such that the vocal sound is less like a real vocal sound of the patient as the degree of return becomes lower and changes the vocal sound of the first avatar such that the vocal sound is more like the real vocal sound of the patient as the degree of return becomes higher.

9. The information processing device according to claim 1, wherein the processing circuitry performs:
outputting a second avatar which is an avatar of a doctor of the clinic in the metaverse via the output interface; and
making an appearance of the second avatar in the metaverse abstract according to the degree of return.

10. The information processing device according to claim 9, wherein the processing circuitry changes the appearance of the second avatar such that the appearance is less like a real appearance of the doctor as the degree of return becomes lower and changes the appearance of the second avatar such that the appearance is more like the real appearance of the doctor as the degree of return becomes higher.

11. The information processing device according to claim 4, wherein the processing circuitry performs:
outputting a first avatar which is an avatar of the patient in the metaverse via the output interface; and
making an external environment viewed from the first avatar in the metaverse abstract according to the degree of return.

12. The information processing device according to claim 11, wherein the processing circuitry changes the external environment such that the external environment is less like the real world as the degree of return becomes lower and changes the external environment such that the external environment is more like the real world as the degree of return becomes higher.

13. An information processing method that is performed by a computer, the information processing method comprising:
acquiring a motion history of a patient who represents themself as an avatar and gets a check-up at a clinic in a metaverse;
determining a specific motion for estimating a state of the patient by analyzing the motion history; and
outputting information based on the specific motion via an output interface.

14. A non-transitory computer-readable storage medium storing a program, the program causing a computer to perform:
acquiring a motion history of a patient who represents themself as an avatar and gets a check-up at a clinic in a metaverse;
determining a specific motion for estimating a state of the patient by analyzing the motion history; and
outputting information based on the specific motion via an output interface.
